(19) United States
(12) Patent Application Publication
(10) Pub. No.: US 2021/0122652 A1
(43) Pub. Date: Apr. 29, 2021

(54) METHOD AND APPARATUS FOR GENERATING ELECTROLYZED WATER

(71) Applicant: Humantek Products Inc., Barrie (CA)

(72) Inventors: Steve Copeland, Barrie (CA); Nicholas Teixeira, Angus (CA)

(73) Assignee: Humantek Products Inc.

(21) Appl. No.: 16/660,919

(22) Filed: Oct. 23, 2019

(51) Int. Cl. C02F 1/461; C02F 9/00; C25B 9/06; C25B 9/20; C02F 1/467

(52) U.S. Cl. CPC C02F 1/4618; C02F 9/005; C25B 9/06; C02F 1/46109; C02F 2201/4616; C02F 1/4674; C02F 2201/003; C02F 2201/4611; C02F 2201/4615; C25B 9/20

(57) ABSTRACT

The present disclosure relates generally to an electrolyzed water generator, kit and method of use. More particularly, the present disclosure relates an electrolyzed water generator with a producing bottle and a solution bottle. Specifically, the present disclosure a method and apparatus of generating electrolyzed water using an apparatus that is able to accurately dose water in a residential or business setting without further devices.

### TECHNICAL FIELD

The present disclosure relates generally to an electrolyzed water generator and method of use. More particularly, the present disclosure relates an electrolyzed water generator with a producing bottle and a solution bottle. Specifically, the present disclosure a method and apparatus of generating electrolyzed water using an apparatus that is able to accurately dose water in a residential or business setting without further devices.

### BACKGROUND

### Background Information

Electrolyzed water is a material that is rising in popularity for its ease of use and lack of any harmful chemicals. Generally, electrolyzed water is classified into three types: acidic electrolyzed water, alkaline ionized water, and alkaline electrolyzed water. Acidic electrolyzed water is naturally germicidal and is used for hygienic purposes to kill microorganisms. Alkaline ionized water is frequently used in drinking water applications while alkaline electrolyzed water is an effective lipid-detergent that may easily wash greasy or oily areas.

Acidic electrolyzed water relies on contact time for its efficacy as a cleaner and sanitizing agent. Traditional generators generally use a combination of cell technology, water, salt, an acid, and electricity. Through these reagents and techniques, a non-toxic, oxidized, antimicrobial solution that is capable of killing many pathogens in less than a minute is created. Sodium ions form sodium hydroxide (NaOH), a strong base that cleans much like a detergent. Chloride ions form hypochlorous acid (HClO), which is a powerful disinfectant. The potent compounds are then rendered harmless either by doing their job by cleaning and disinfecting or they are simply rendered inactive over time.

### SUMMARY

As a result, of the rendering ineffective of the compounds, there is a need for a method and apparatus for generating electrolyzed water that is usable in a residential or business scenario.

In one aspect, an exemplary embodiment of the present disclosure may provide an electrolyzed water producing apparatus comprising: a solution bottle with a visual indictor line, a producing bottle with a further visual indicator line, an electrolysis unit comprising, at least one anode, at least one cathode; and a power supply operatively electrically connected to the at least one anode and the at least one cathode. This exemplary embodiment or another exemplary embodiment may provide the solution bottle further comprises a bottle portion with the visual indicator line; and a cap portion with two recesses. This exemplary embodiment or another exemplary embodiment may provide a first recess operative to measure an effective amount of salt to add to an acid filled in the solution bottle to a visual indicator line; and a second recess operative to dose an amount of a solvent in the producing bottle with the contents of the solution bottle after acid and salt have been mixed to a homogeneous consistency. This exemplary embodiment or another exemplary embodiment may provide that the solution bottle contains more than one dose. This exemplary embodiment or another exemplary embodiment may provide the at least one anode comprises one anode and the at least one cathode comprises two cathodes. This exemplary embodiment or another exemplary embodiment may provide two cathodes are made of uncoated titanium mesh. This exemplary embodiment or another exemplary embodiment may provide the anode is made of a coated titanium mesh anode. This exemplary embodiment or another exemplary embodiment may provide a body which supports the electrolysis unit and an integrally formed compartment for a spray assembly to attach to the producing bottle. This exemplary embodiment or another exemplary embodiment may provide a power button operative to permit electrical flow from the power supply to the at least one anode and at least one cathode. This exemplary embodiment or another exemplary embodiment may provide the acid is vinegar, the solvent is tap water and the salt is coarse table salt.

In another aspect, an exemplary embodiment of the present disclosure may provide for an all-in-one kit for producing electrolyzed water comprising: an electrolysis unit comprising: at least one anode; at least one cathode; and a power supply operatively electrically connected to the at least one anode and the at least one cathode; a producing bottle with an engagable connection operative to engage with the electrolysis unit; and a spray assembly a solution bottle comprising: a bottle portion; and a cap portion with two recesses to properly measure liquid and powders.

In yet another aspect, an exemplary embodiment of the present disclosure may provide for a method for making electrolyzed water comprising: measuring an acid to a visually indicated line on a solution bottle, measuring a salt to a visually indicated level on a first recess of a cap of the solution bottle, adding the salt to the acid in the solution bottle, attaching the cap of the solution bottle to the solution bottle, agitating the salt and acid to combine the salt and acid into a homogeneous mixture, removing the cap of the solution bottle from the solution bottle, pouring a portion of the homogeneous mixture into a second recess of the cap, filling a solvent to a visually indicated line on a producing bottle, adding the homogeneous mixture from the second recess of the cap to the producing bottle to create a solution, attaching the producing bottle to an electrolysis unit, and activating at least one anode and at least one cathode on the electrolysis unit in contact with the solution in the producing bottle thereby producing electrolyzed water. This exemplary embodiment or another exemplary embodiment may provide the salt is sodium chloride. This exemplary embodiment or another exemplary embodiment may provide the acid is acetic acid. This exemplary embodiment or another exemplary embodiment may provide the solvent is water. This exemplary embodiment or another exemplary embodiment may provide wherein after activating comprises: removing the producing bottle from the electrolysis unit, and attaching a sprayer assembly to the producing bottle. This exemplary embodiment or another exemplary embodiment may provide after activating comprises: using the electrolyzed water, pouring a further portion of the homogeneous mixture into the second recess of the cap, filling the solvent to a visually indicated line on the producing bottle, adding the homogeneous mixture from the second recess of the cap to the producing bottle, attaching the producing bottle to the electrolysis unit, and activating the at least one anode and the at least one cathode on the electrolysis unit thereby producing further electrolyzed water. This exemplary embodiment or another exemplary embodiment may provide repeating the steps of using through activating until the homogeneous mixture is exhausted; and measuring the acid to visually indicated line on the solution bottle, measuring the salt to the visually indicated level on the first recess of the cap of the solution bottle; adding the salt to the acid in the solution bottle, attaching the cap of the solution bottle to the solution bottle, agitating the salt and acid to combine the salt and acid into an additional homogeneous mixture. This exemplary embodiment or another exemplary embodiment may provide depressing an actuator, applying electricity to the at least one anode and the at least one cathode in contact with the solution in the producing bottle, converting the solution to at least one further acid and at least one base. This exemplary embodiment or another exemplary embodiment may provide the further acid is hypochlorous acid and at sodium hydroxide is least one of the bases.

### BRIEF DESCRIPTION OF THE SEVERAL VIEWS OF THE DRAWINGS

A sample embodiment of the disclosure is set forth in the following description, is shown in the drawings and is particularly and distinctly pointed out and set forth in the appended claims. The accompanying drawings, which are fully incorporated herein and constitute a part of the specification, illustrate various examples, methods, and other example embodiments of various aspects of the disclosure. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. In some examples, an element shown as an internal component of another element may be implemented as an external component and vice versa. Furthermore, elements may not be drawn to scale.

FIG. 1 (FIG. 1) is a front right perspective view of an apparatus for producing electrolyzed water.

FIG. 2 (FIG. 2) is a front right exploded view of the apparatus for producing electrolyzed water.

FIG. 3 (FIG. 3) is a right side cross sectional view along line 3-3 in FIG. 1 showing the internal workings of the apparatus.

FIG. 4A (FIG. 4A) is a front right perspective view of a solution bottle bottom portion.

FIG. 4B (FIG. 4B) is a front right perspective view of a solution bottle top along line 4B-4B in FIG. 1.

FIG. 5A (FIG. 5A) is a side elevational view of a disassembled solution bottle with salt being added from a first recess.

FIG. 5B (FIG. 5B) is a side elevational view of a disassembled solution bottle with solution being poured out into a second recess.

FIG. 5C (FIG. 5C) is a side elevational view of a producing bottle 14 with solution being added to it from the second recess of the solution bottle.

Similar numbers refer to similar parts throughout the drawings.

### DETAILED DESCRIPTION

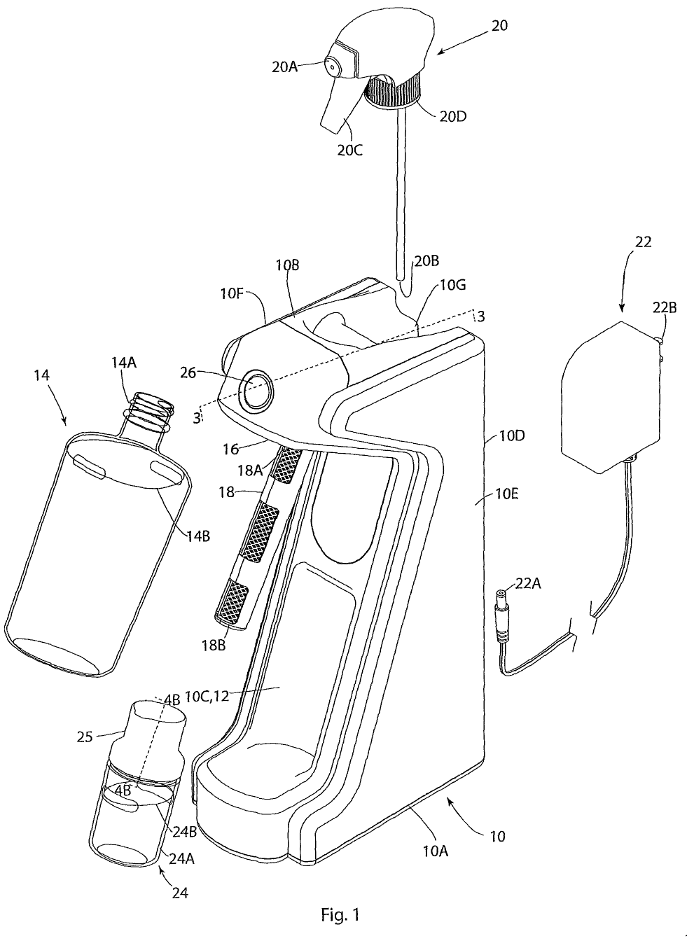

A new apparatus 10 and method producing electrolyzed water using the apparatus 10 is depicted in the present disclosure and throughout FIGS. 1-4D. Apparatus 10 is a new and improved apparatus for producing electrolyzed water, as will be discussed hereafter.

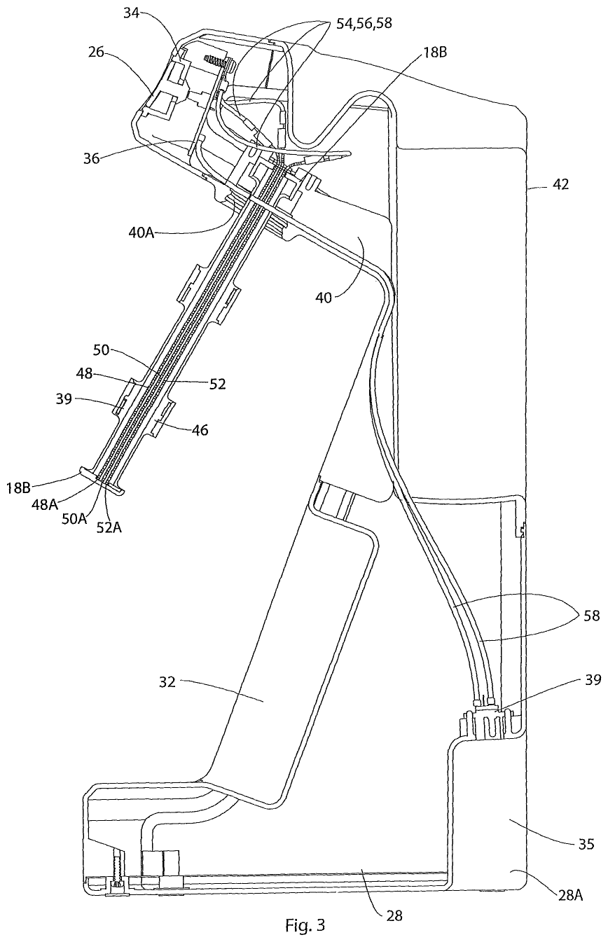

Referring specifically to FIG. 1, a front right perspective view of an apparatus 10 for producing electrolyzed water is shown. The apparatus 10 has a body with a base 10A, a top 10B that is longitudinally opposed to the base 10A, a front 10C and a back 10D that is transversely opposed to the front 10C and a first side 10E and a second side 1OF that is horizontally opposed to the first side 10E. The base 10A and back 10D are generally flat and disposed at right angles to one another. Additional views of these exemplary body sections will be seen with respect to FIG. 3.

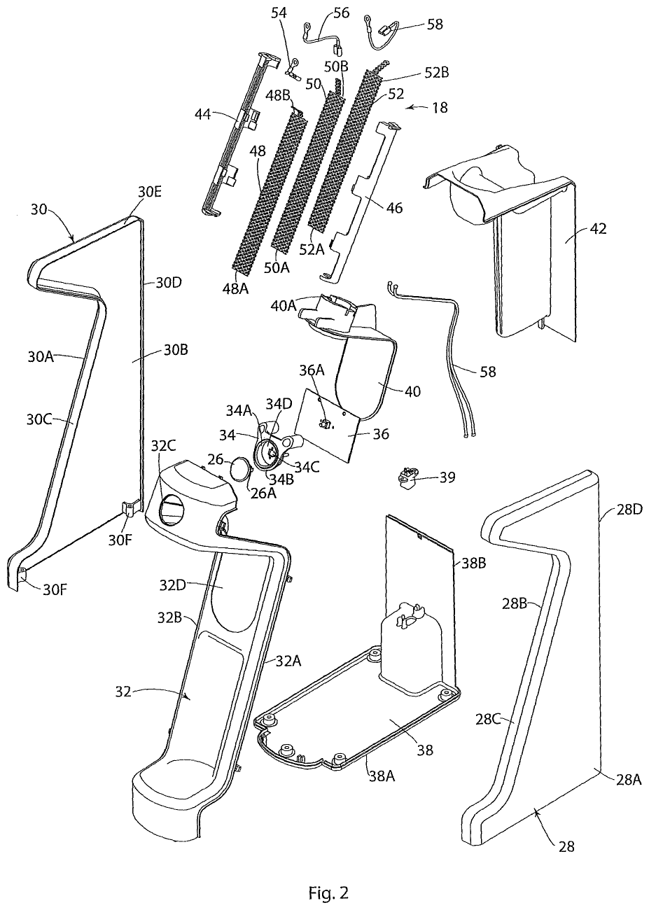

The front 10C has a recessed section 12 operative to accept a producing bottle 14. The recessed section 12 is generally angled within the front 10C from a base 12A to a side 12B. In one embodiment, the angle is greater than 90 degrees but less than 180 degrees. In a further embodiment the angle is greater than 90 degrees but less than 150 degrees. The side 12B of the recessed section 12 is generally concave in nature in order to accept the producing bottle 14. Further embodiments have been contemplated depending on the ultimate shape of the producing bottle 14. Adjacent to the recessed section 12 at a top 12C of its side 12B is an angled portion 16. The angled portion 16 is generally at an angle of approximate 90 degrees to the side 12B of the recessed section. The angled portion 16 contains an electrode assembly 18 with a body that is generally cylindrical in nature with a first end 18A and a second end 18B as will be discussed later with respect to FIG. 2 and FIG. 3. Further, there is a spray assembly 20. The spray assembly 20 has an output 20A, a bottom 20B with an actuator 20C and a threaded cap 20D. The output 20A is in fluid connection with the bottom 20B. The spray assembly 20 is operative to nest within an aperture 10G of the apparatus 10 for storage.

In this embodiment, the producing bottle 14 has a body that is generally bulbous in shape and has a top threaded portion 14A and a line 14B. The top threaded portion 14A may be any such connection member and is not limited to threads. The line 14B is operative to indicate a level that the producing bottle 14 should be filled. In an exemplary embodiment the producing bottle 14 is filled with a solvent. In one embodiment, the solvent is water. Water may include, bottled water, distilled water, reverse osmosis water, or tap water. In one embodiment the line 14B is at 400-600 mL. In a further embodiment the line is at 500 mL. This will be discussed later with respect to the method. It should be noted that the solvent is never within the body of the apparatus 10. Additionally, the producing bottle 14 can comprise any suitable material that allows it to function as described herein. In an exemplary embodiment the producing bottle 14 may be made of such materials including one or more: plastics, glass, polymers, ceramics, synthetic materials, metals, or metal alloys (e.g., stainless steel, steel, carbon steel, titanium, and/or any other suitable metal).

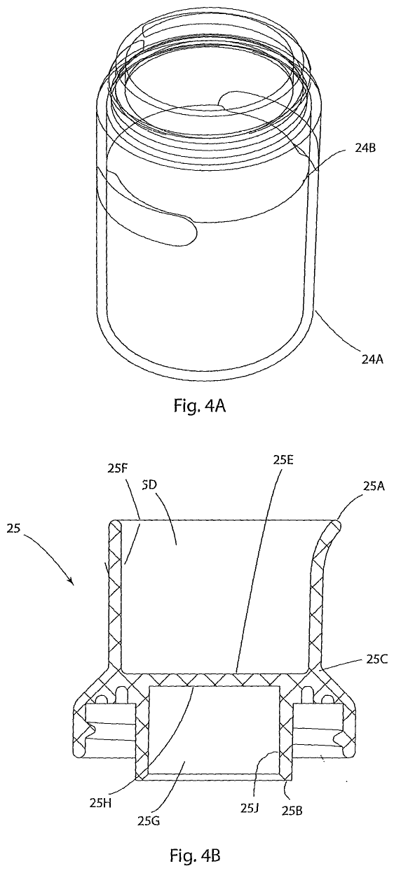

The apparatus further includes a power adapter 22 with a first end 22A that interfaces with a connection on the apparatus 10 and a second end 22B that is adapted to interface with a wall outlet (not shown). The first end 22A and the second end 22B are electrically connected. Separate from the apparatus 10 is an assembled solution bottle 24 that will be discussed later with respect to FIG. 4A and FIG. 4B. Further seen is a power button 26 located on the front side 10C of the apparatus 10.

Referring specifically to FIG. 2 and FIG. 3, an exploded view and a cross-sectional view of the pieces that make up the exemplary apparatus 10 are shown. Specifically, the pieces are shown in an exemplary way to be assembled. These pieces are but one embodiment and other configurations have been contemplated. In the shown embodiment, a left side wall 28 that has an outer surface 28A that corresponds to the first side 10E while a right side wall 30 has an outer surface 30A that corresponds to the second side 10F. While the left side wall 28 further includes an inside wall 28B as well as first side bevel 28C, a second side bevel 28D and a top side bevel 28E. The right side wall 30 further includes an inside wall 30B as well as a first side bevel 30C, a second side bevel 30D and a top side bevel 30E. The left side wall 28 further includes a plurality of engagable apertures 28F while the right side wall 30 further includes a plurality of engagable apertures 30F.

Further there is a front piece 32 that corresponds to the front 10C and a portion of the top 10B. The front piece 32 has a first side engageable portion 32A and a second side engageable portion 32B. The first side engageable portion 32A engages with the first side bevel 28C of the left side wall 28 to fit in nestable engagement therewith. The second side engagable portion 32B engages with the right side 30 with the first side bevel 30C to fit in nestable engagement therewith. The front piece 32 further includes an aperture 32C. The power button 26 is operative to nest within the aperture 32C. The power button 26 further sits in a housing 34. The housing 34 has a circular aperture 34A operative to accept the power button 26 at a first side 34B and engage with an electronic member 34C at the rear 34D of the housing. The power button 26 has a projection 26A that passes through the rear 34D of the housing 34 and is in operative connection with a projection 36A on a printed circuit board 36.

Further included is a base 38. The base 38 has a bottom portion 38A that is commiserate with the base 10A of the apparatus along with a back portion 38B commiserate with a portion of the back 10D. The back portion 38B further includes a plurality of recesses 38C along with an aperture 38D. The aperture 38D is operative to receive a power connector 39 The bottom portion 38A of the base 38 further includes a plurality of engagable apertures 38E. The bottom portion 38A and back portion 38B are operative to interface with the left side wall 28 and right side wall 30 along with the front piece 32 in order to be placed into operative engagement with one another.

Further included is a top piece 40. The top piece 40 is generally L-shaped and is placed into a bottle support aperture 32D of the front piece 32. The top piece 40 is commiserate with a portion of the recessed section 12 proximate the top 12C of its side 12B and the angled portion 16. The top piece has a threaded portion 40A operative to accept the threads 14A of the producing bottle 14. Additionally, there is a top spray head holder 42. The top spray head holder 42 encompasses the aperture 10G as well as a portion of the back 10D. The top spray head holder 42 engages with the base 38.

The electrode assembly 18 includes a left bracket 44 and a right bracket 46 operative to hold all three electrodes in place. The three electrodes include a first electrode 48, a second electrode 50, and a third electrode 52 in place. Each electrode has a body with a first end 48A, 50A, 52A and a second end 48B, 50B, 52B longitudinally disposed thereof. Proximate the second end 48B, 50B, 52B, is a connection 48C, 50C, 52C to wiring 54, 56, 58. The connection 48C of the first electrode connects to the wiring 54. The connection 50C of the second electrode connects to the wiring 56. The connection 52C of the third electrode connects to the wiring 58. In one embodiment, the first electrode 48 is an uncoated titanium mesh cathode, the second electrode 50 is a coated titanium mesh anode and the third electrode 52 is an uncoated titanium mesh cathode. The wiring 54, 56, 58 are further connected to the printed circuit board 36. There is additional wiring 58, that allows the power to be connected to the circuit board 36.

While the body of the electrodes 48, 50, 52 is shown to be a strip like architecture, the electrodes 48, 50, 52, also have any suitable shape that allows them to function as electrodes. In some embodiments, the electrodes may comprise one or more wires, plates, rods, meshes, blocks, or screens. In some embodiments the electrodes 48, 50, 52 can comprise any suitable material that allows them to function as described herein to form electrolyzed water. These suitable electrode materials include, but are not limited to, at least one or more of the following: platinum, stainless steel, coated stainless steel, dimensionally stable anode materials, ruthenium coated on a conductive material, ruthenium oxide coated titanium, lead, tungsten, tungsten carbide, titanium diboride, nickel, cobalt, nickel tungstate, nickel titanate, graphite, ceramic electrode material, platinum, silver, titanium carbide, a porous electrode material, and a foamed electrode material.

In some embodiments the power adapter 22 provides electricity to the three electrodes 48, 50, 52 to cause electrolysis within the cell when the electrode assembly 18 may be actuated by the power button 26. In further embodiments the power adapter 22 is then configured to automatically provide suitable electrical current and flow allowing the amperage to reach a set limit and/or voltage within the producing bottle 14. As such, the system is configured to monitor and adjust in near real time, intermittently, constantly, or in any other suitable manner the amount of electrical current and flow that is added to the producing bottle 14 to create a desired voltage within the cell. The electrical current may be varied so as to compensate for fluctuations in fluid conductivity. In a further embodiment, the power adapter is configured to keep the amperage supplied in a substantially constant manner. In yet another embodiment, the system is configured to autonomously modify the amperage provided to the electrode assembly 18 in order to function optimally and to produce one or more desired products by driving the reaction towards or away from an equilibrium.

Referring now to FIGS. 4A and 4B, views of portions of the solution bottle 24 are shown. The solution bottle 24 has a bottle portion 24A and a cap portion 25 that is engagably attached therewith. In one embodiment the attachment may occur by a set of male and female threads. In a further embodiment it may be a snap fitting. The bottle portion 24A further includes a line 24B located on it that circumscribes the bottle portion 24A. In an exemplary embodiment, an acid is filled to the line 24B. In one particular embodiment, the acid is acetic acid, or vinegar. In one embodiment the line is located at about 50 mL. In a further embodiment the fill marker is at 49.3 ml.

Referring specifically to FIG. 4B, the cap portion 25 has a body with a top 25A, a bottom 25B that is longitudinally disposed from the top 25A and an outside surface 25C that is formed around the top 25A and the bottom 25B. At the top 25A of the cap portion 25 there is a first recess 25D. The first recess 25D extends within the cap portion and is bounded by a terminus 25E and a first interior surface 25F. The first recess 25D of the cap portion 25 in one embodiment may be operative to be filled with an effective amount of salt. In one embodiment the first recess 25D holds about 10 g to about 30 g of salt. In a further embodiment the recess holds 20 g of sodium chloride salt. In one embodiment this salt is sodium chloride in the form of coarse pickling salt. The salt is filled within the first recess 25D until the first recess 25D is full and begins to exceed the top 25A. The size of the first recess 25D is so designed to act as portion of salt to keep ratios proper within the solution bottle 24 as a whole. For example, the solution bottle 24 may be filled to its line 24B with an acid and the first recess 25D may be filled with a salt to the top 25A. The ratio of the salt to the acid would be proper based on the size of the first recess 25D and the level of the line 24B. This will be discussed later with respect to the operation.

The cap portion 25 further has a second recess 25G at the bottom 25B. The second recess 25G extends within the cap portion and is bounded by a base 25H and a second interior surface 25J. The second recess 25G is operative to be filled with an effective amount of solution as will be discussed later with respect to the operation. The second recess 25H may be filled multiple times with the effective amount of the solution. In one embodiment the recess holds about 10% of the volume of the solution bottle 24 when filled to its line 24B. In one embodiment the recess holds about 5% of the volume of the solution bottle 24 when filled to its line 24B. In one embodiment the recess holds about 20% of the volume of the solution bottle 24 when filled to its line 24B.

Having thus described an exemplary non-limiting configuration of the apparatus 10, its operation will be discussed with reference to some exemplary features used with the various embodiments.

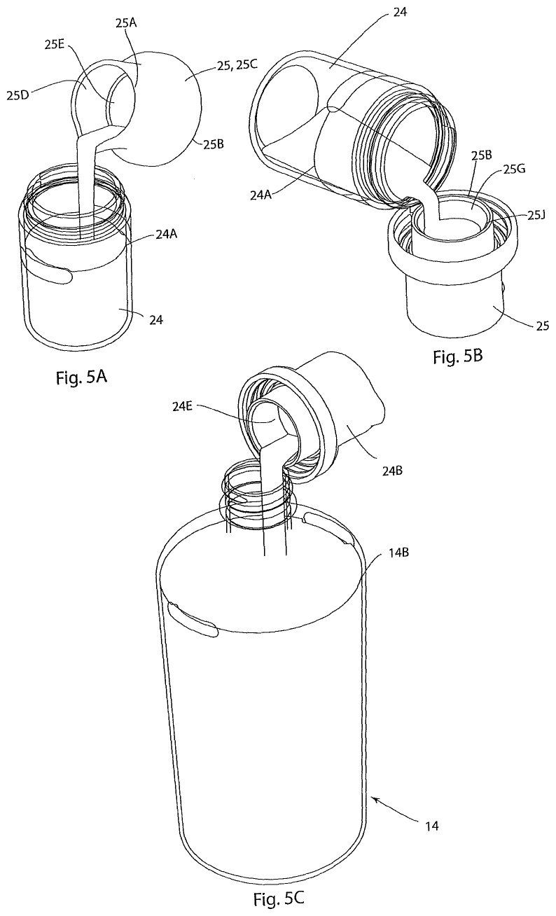

Referring now to FIG. 5A, the acid is filled up to the line 24B of the solution bottle 24. Then, a salt from the first recess 25D of the cap portion 25 is added to the bottle portion 24A. The entirety of the assembled solution bottle 24 is then vigorously shaken in order to dissolve the salt within the acid and create a homogeneous mixture of salt interspersed with the acid.

Referring specifically to FIG. 5B, after the salt is dissolved in the acid, the cap portion 25 is then removed from the solution bottle 24 and the cap is inverted so that the second recess 25G is open and the first recess 25D is placed towards a surface. and able to accept an amount of homogenous mixture of salt interspersed with acid. The homogenous mixture of salt interspersed with acid is poured from the solution bottle 24 into the second recess 25G of the cap 25. The second recess 25G is so designed that it may contain a measured portion of the homogenous mixture of salt interspersed with acid when the recess 25G is filled up to the bottom 25B. As has been discussed earlier, the solution bottle 14 may hold further portions of the homogenous mixture of salt interspersed with acid to create further measured portions of the homogenous mixture of salt interspersed with acid for future use.

Referring now to FIG. 5C, the producing bottle 14 having been filled up with a solvent up to the line 14B has the measured portion of the homogenous mixture of salt added to the producing bottle 14. By doing so, the salt, acid and solvent are now combined and ready to be electrolyzed. The threads 14A of the producing bottle are attached to the threaded portion 40A of the apparatus. The power button 26 is depressed which is operative to allow power to flow to the electrode assembly 18 by actuating the printed circuit board 26 to permit the flow of electricity to the electrode assembly 18 and to the individual electrodes 48, 50, 52, now within the producing bottle 14. As such, the solution is electrolyzed. This occurs at the anode 50 by the chlorine ion Cl⁻ of a salt, in this embodiment, sodium chloride, by the following reactions:

NaCl→Na⁺+Cl₂

2Cl⁻→Cl₂+2e⁻

As can be seen, this reaction at the anode produces chlorine gas (Cl₂). Then, this chlorine gas is dissolved into the subject solvent, in this embodiment, water and is converted into hypochlorous acid (HClO) through a reaction represented by a following reaction:

Cl₂+H₂O→H⁺+Cl⁻+HClO.

The concentration of any residual chlorine, or in the alternative the amount of chlorine generated is governed by a product of the electric current flowing between the electrodes, and by the amount of chlorine ion (Cl⁻) added.

On the other hand, at the cathodes 50, 52 sides, water is electrolyzed to generate hydrogen gas (H₂) and hydroxide ion (OH⁻). Furthermore, the sodium ion (Na⁺) from the salt reacts with the hydroxide ion (OH⁻) to reversibly produce sodium hydroxide (NaOH) by the reactions:

2H₂O+4e⁻→H₂+2OH⁻

Na⁺+OH⁻→NaOH

As a result, the now electrolyzed solution includes both hypochlorous acid (HClO) and sodium hydroxide to act as a sanitizer and cleaner, respectively.

While sodium chloride has been used as an example further embodiments may provide for a different salt to be used. To that end, instead of sodium chloride, any suitable alkali salt that is capable of allowing the apparatus 10 to produce electrolyzed water may be used. Some examples of suitable non-sodium chloride may include, but are not limited to, sodium carbonate (Na₂CO₃), soda ash, sodium bicarbonate (NaHCO₃), potash, potassium carbonate (K₂CO₃), potassium bicarbonate (KHCO₃), sodium chloride, potassium nitrate (KNO₃), potassium chloride (KCl), potassium chlorate (KClO₃), sodium phosphate, and/or any other suitable electrolyte (e.g., any suitable alkali ion containing electrolyte). Depending on the salt used, the size of the first recess 25D and second recess 25G may differ. Further, the lines 14B and 24B may also differ as the amount of acid or solvent may further change.

In some cases, after the apparatus 10 has produced a desired amount of electrolyzed water, the apparatus can be shut down in any suitable manner and for any suitable reason. In some embodiments this may include interrupting or stopping the flow of electricity to electrode assembly 18. This could include, but is not limited to, disconnecting the second end 22B of the power adapter 22 from a power source or re-actuating the power button 26 in order to stop electrical flow. The apparatus 10 may then be started back up at and desired time when more electrolyzed water is desired to be made.

The mixing bottle 14 may then be outfitted with the spray assembly 20 and the spray assembly may be actuated and used to disperse the sanitizer and cleaner onto a surface. When the mixing bottle 14 is empty, the process may be repeated with the remaining doses with the solution bottle 24. Once the solution bottle 24 is depleted, it may be remade with the salt and acid solution. The mixing bottle 14 may be used virtually indefinitely eliminating the need for additional plastic waste when compared to prior art that may sell premixed acid and salt solutions.

The apparatus 10 further reduces the need to carefully measure various chemicals. Other devices require that a user use their own spoons, cups or other marked devices to add to the water. This will result in in inconsistent solution levels of hypochlorous acid (sanitizer) and sodium hydroxide (cleaner) and result in ineffective cleaning or sanitizing of surfaces and other places desired to be disinfected. After production, the electrolyzed water may then be applied to any surface desiring to be cleaned. The electrolyzed water can be applied at any suitable time as a user deems fit.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. For example, embodiments of technology disclosed herein may be implemented using hardware, software, or a combination thereof. When implemented in software, the software code or instructions can be executed on any suitable processor or collection of processors, whether provided in a single computer or distributed among multiple computers. Furthermore, the instructions or software code can be stored in at least one non-transitory computer readable storage medium.

Also, a computer or smartphone utilized to execute the software code or instructions via its processors may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers or smartphones may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The various methods or processes outlined herein may be coded as software/instructions that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, USB flash drives, SD cards, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the disclosure discussed above. The computer readable medium or media can be transportable, such that the program or programs stored thereon can be loaded onto one or more different computers or other processors to implement various aspects of the present disclosure as discussed above.

The terms “program” or “software” or “instructions” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

All definitions, as defined and used herein, should be understood to control over dictionary definitions, definitions in documents incorporated by reference, and/or ordinary meanings of the defined terms.

“Logic”, as used herein, includes but is not limited to hardware, firmware, software, and/or combinations of each to perform a function(s) or an action(s), and/or to cause a function or action from another logic, method, and/or system. For example, based on a desired application or needs, logic may include a software controlled microprocessor, discrete logic like a processor (e.g., microprocessor), an application specific integrated circuit (ASIC), a programmed logic device, a memory device containing instructions, an electric device having a memory, or the like. Logic may include one or more gates, combinations of gates, or other circuit components. Logic may also be fully embodied as software. Where multiple logics are described, it may be possible to incorporate the multiple logics into one physical logic. Similarly, where a single logic is described, it may be possible to distribute that single logic between multiple physical logics.

Furthermore, the logic(s) presented herein for accomplishing various methods of this system may be directed towards improvements in existing computer-centric or internet-centric technology that may not have previous analog versions. The logic(s) may provide specific functionality directly related to structure that addresses and resolves some problems identified herein. The logic(s) may also provide significantly more advantages to solve these problems by providing an exemplary inventive concept as specific logic structure and concordant functionality of the method and system. Furthermore, the logic(s) may also provide specific computer implemented rules that improve on existing technological processes. The logic(s) provided herein extends beyond merely gathering data, analyzing the information, and displaying the results. Further, portions or all of the present disclosure may rely on underlying equations that are derived from the specific arrangement of the equipment or components as recited herein. Thus, portions of the present disclosure as it relates to the specific arrangement of the components are not directed to abstract ideas. Furthermore, the present disclosure and the appended claims present teachings that involve more than performance of well-understood, routine, and conventional activities previously known to the industry. In some of the method or process of the present disclosure, which may incorporate some aspects of natural phenomenon, the process or method steps are additional features that are new and useful.

The articles “a” and “an,” as used herein in the specification and in the claims, unless clearly indicated to the contrary, should be understood to mean “at least one.” The phrase “and/or,” as used herein in the specification and in the claims (if at all), should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper”, “above”, “behind”, “in front of”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal”, “lateral”, “transverse”, “longitudinal”, and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed herein could be termed a second feature/element, and similarly, a second feature/element discussed herein could be termed a first feature/element without departing from the teachings of the present invention.

An embodiment is an implementation or example of the present disclosure. Reference in the specification to “an embodiment,” “one embodiment,” “some embodiments,” “one particular embodiment,” or “other embodiments,” or the like, means that a particular feature, structure, or characteristic described in connection with the embodiments is included in at least some embodiments, but not necessarily all embodiments, of the invention. The various appearances “an embodiment,” “one embodiment,” “some embodiments,” “one particular embodiment,” or “other embodiments,” or the like, are not necessarily all referring to the same embodiments.

If this specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0. % of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Additionally, any method of performing the present disclosure may occur in a sequence different than those described herein. Accordingly, no sequence of the method should be read as a limitation unless explicitly stated. It is recognizable that performing some of the steps of the method in a different order could achieve a similar result.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures.

In the foregoing description, certain terms have been used for brevity, clarity, and understanding. No unnecessary limitations are to be implied therefrom beyond the requirement of the prior art because such terms are used for descriptive purposes and are intended to be broadly construed.

Moreover, the description and illustration of various embodiments of the disclosure are examples and the disclosure is not limited to the exact details shown or described.

What is claimed:

1. A kit for producing electrolyzed water comprising:
an electrolysis unit comprising:
at least one anode;
at least one cathode; and
a power supply operatively electrically connected to the at least one anode and the at least one cathode;
a producing bottle with an engagable connection operative to engage with the electrolysis unit; and
a solution bottle with a visual indictor line.

2. The kit of claim 1, wherein the solution bottle further comprises:
a bottle portion with the visual indicator line; and
a cap portion formed with two recesses.

3. The kit of claim 2, wherein two recesses include:
a first recess, wherein the first recess is adapted to measure an effective amount of salt to add to an acid filled in the solution bottle to a visual indicator line; and
a second recess, wherein the second recess is adapted to measure an effective amount of salt and acid to dose an amount of a solvent in the producing bottle with the contents of the solution bottle after acid and salt have been mixed to a homogeneous consistency.

4. The kit of claim 3, wherein the solution bottle is adapted to contain more than one dose.

5. The kit of claim 1, wherein the at least one anode comprises one anode and the at least one cathode comprises two cathodes.

6. The kit of claim 5, wherein two cathodes are made of uncoated titanium mesh.

7. The kit of claim 6, wherein the anode is made of a coated titanium mesh anode.

8. The kit of claim 1, wherein the kit further comprises:
a body which supports the electrolysis unit and an integrally formed compartment for a spray assembly to attach to the engagable connection of the producing bottle when the producing bottle is not engaged with the electrolysis unit.

9. The kit of claim 1, further comprising a power button operative to permit electrical flow from the power supply to the at least one anode and at least one cathode.

10. The kit of claim 3, wherein the acid is vinegar, the solvent is tap water and the salt is coarse table salt.

11. A method for making electrolyzed water comprising:
measuring an acid to a solution bottle;
measuring a salt to a visually indicated level on a first recess of a cap of the solution bottle;
adding the salt to the acid in the solution bottle;
attaching the cap of the solution bottle to the solution bottle;
agitating the salt and acid to combine the salt and acid into a homogeneous mixture;
removing the cap of the solution bottle from the solution bottle;
pouring a portion of the homogeneous mixture into a second recess of the cap;
filling a solvent into a producing bottle;
adding the homogeneous mixture from the second recess of the cap to the producing bottle to create a solution;
attaching the producing bottle to an electrolysis unit; and
activating at least one anode and at least one cathode on the electrolysis unit in contact with the solution in the producing bottle thereby producing electrolyzed water.

12. The method of claim 11, wherein the step of measuring the acid includes: filling the solution bottle with acid to a visually indicated line.

13. The method of claim 12, wherein the step of filling the solvent includes: filling the producing bottle with a solvent to a visually indicated line.

14. The method of claim 11, further comprising:
cleaning a surface with the electrolyzed water.

15. The method of claim 11, wherein the salt is sodium chloride, the acid is acetic acid and the solvent is water.

16. The method of claim 11, wherein after activating comprises:
removing the producing bottle from the electrolysis unit; and
attaching a sprayer assembly to the producing bottle.

17. The method of claim 13, wherein after activating comprises:
using the electrolyzed water;
pouring a further portion of the homogeneous mixture into the second recess of the cap;
filling the solvent to a visually indicated line on the producing bottle;
adding the homogeneous mixture from the second recess of the cap to the producing bottle;
attaching the producing bottle to the electrolysis unit; and
activating the at least one anode and the at least one cathode on the electrolysis unit thereby producing further electrolyzed water.

18. The method of claim 14, further comprising:
repeating the steps of using through activating until the homogeneous mixture is exhausted; and
measuring the acid to visually indicated line on the solution bottle;
measuring the salt to the visually indicated level on the first recess of the cap of the solution bottle;
adding the salt to the acid in the solution bottle;
attaching the cap of the solution bottle to the solution bottle;
agitating the salt and acid to combine the salt and acid into an additional homogeneous mixture.

19. The method of claim 11, wherein activating further includes:
depressing an actuator;
applying electricity to the at least one anode and the at least one cathode in contact with the solution in the producing bottle,
converting the solution to at least one further acid and at least one base.

20. The method of claim 19, wherein the further acid is hypochlorous acid and at sodium hydroxide is least one of the bases.
